(19) United States
(12) Patent Application Publication
(10) Pub. No.: US 2007/0015211 A1
(43) Pub. Date: Jan. 18, 2007

(54) Conformer-specific antibodies and method of use, thereof

(75) Inventors: Vishwanath Lingappa, San Francisco, CA (US); Mahadevappa Dharma Prasad, San Francisco, CA (US); William Hansen, San Francisco, CA (US); Joshua Lehrer-Graiwer, San Francisco, CA (US); Carsten Korth, Dusseldorf (DE)

Correspondence Address: FOLEY & LARDNER LLP, 1530 PAGE MILL ROAD, PALO ALTO, CA, 94304, US

(73) Assignee: Prosetta Corporation

(21) Appl. No.: 11/473,460

(22) Filed: Jun. 22, 2006

Related U.S. Application Data
(60) Provisional application No. 60/693,136, filed on Jun. 22, 2005.

(51) Int. Cl. G01N 33/53; C07H 21/04; C12P 21/06; C12N 5/06; C07K 16/18

(52) U.S. Cl. 435/7.1; 435/69.1; 435/320.1; 435/326; 530/387.1; 536/23.53

(57) ABSTRACT

A number of polypeptides exist in different three-dimensional conformations, or “conformers,” which have different biological functions despite having the same or substantially the same primary polypeptide sequences. The existence of different polypeptide conformers underlies to a variety of disease states, which may be assessed using antibody that is specific for a given conformer. Thus, such antibody thus is useful for identifying or detecting certain conformers within a mixed population of conformers.

### CROSS-REFERENCE TO RELATED APPLICATIONS

This application claims the benefit under 35 U.S.C. 119(e) of U.S. 60/693,136, filed Jun. 22, 2005.

### FIELD OF THE INVENTION

The invention provides a method for producing a polypeptide conformer-specific antibody useful for identifying or detecting a particular polypeptide conformer present in a mixed population of conformers, as well as the conformer-specific antibody produced by the method of the invention.

### BACKGROUND

Polypeptides are more heterogeneous than generally recognized, with multiple folded forms being produced in cells, each of which can be subsequently modified in different ways. Examples of such modifications include but are not limited to covalent post-translational modifications (e.g., glycosylation, acylation, prenylation, farnesylation, etc.) and proteolytic cleavage. Polypeptide modifications are also known to occur under different environmental conditions and in different compartments of cells [e.g., the endoplasmic reticulum (ER), Golgi apparatus, and endosomes]. Likely as a result of thermodynamics considerations, differently folded forms of a polypeptide, termed conformers, are not spontaneously interconvertible under standard physiological conditions.

Recent data suggest that different conformers have different biological functions and that cells regulate the production of different conformers, to influence cellular functions such as signaling and metabolism. Changes in the relative amounts of different conformers of a subject polypeptide have been shown to be associated with disease and may be important in determining whether certain pharmaceutical compositions produce beneficial effects, undesirable side effects, or the relative amounts, thereof.

Detecting the presence of different polypeptide conformers in a biological sample has traditionally been difficult and remains a challenge. For those few polypeptide conformers that happen to differ in transmembrane topology, it may be possible to distinguish conformers based on the orientation of the polypeptide within a membrane. However, the majority of polypeptides existing as different conformers cannot be distinguished by differential membrane topology, and other identification methods must be found. Moreover, even for those that do exist in different topological forms, scoring of topology requires tools such as protease digestion that are painstaking, labor and time intensive, and substantially less sensitive than antibody-based assays. Conformer-specific chemical modification is one promising approach, but the opportunities for selective chemical modification of certain conformers is limited and unpredictable.

A critical problem for analyzing conformers is that the most precise analytical tools are also the most perturbing of the system being studied. For example, crystallographic structure analyses requires that a protein be purified. Purification often results in the loss of a large percentage of the polypeptide present in the starting material, which fraction may comprise conformers of interest. Moreover, the process of crystal formation may actually exclude the conformers that are of most interest, resulting in homogeneous crystals that do not accurately reflect the different conformers that of a polypeptide that exist in cells.

Furthermore, the artificial conditions and solvents required for crystallization may alter the energy barrier between conformers driving conformer interconversion that would rarely happen and/or be biologically unimportant under physiological conditions where conformers have a shorter half life, bind to other proteins, and are subject to other conditions that stabilize their conformer structure.

Antibodies can be used as conformer-specific reagents; however, the production of such antibodies can be a laborious and painstaking task and not all antibodies are useful for distinguishing between conforms. Typically when one wishes to raise an antibody, three options are available.

First, a polyclonal antibody can be raised (e.g., in a rabbit or goat). This approach has the advantage of generating a heterogeneous mixture of individual antibodies, which often provides robust antigen recognition due to the contribution of multiple epitopes. At the same time, polyclonal antibodies represent a limited (i.e., finite) resource that is heterogeneous in nature, and constantly changing with additional boosts and bleeds, leading to inconsistent experimental results.

Second, a monoclonal antibody (MAb) can be produced. This approach is superior when one requires a defined, homogenous reagent of ostensibly infinite supply. However, the generation of MAbs is a more difficult task, necessitating cell fusion and screening of hybridomas.

Finally, phage display libraries can be used to generate antibodies and to carry out their in vitro affinity maturation through sequential selection. Such methods are plagued with their own difficulties.

The present inventors have developed a general methodology that allows polyclonal antisera, for which the source animal is still alive or B-cell mRNA is available, to be used as the source for monoclonal antibodies specific to particular polypeptide conformers.

### SUMMARY OF THE INVENTION

One embodiment of the invention provides a method for producing an antibody specific for a particular polypeptide conformer (i.e., a conformer of interest), the method comprising:

(a) providing a polypeptide composition enriched for a particular polypeptide conformer;

(b) administering to an animal the polypeptide composition of (a) to produce a polyclonal antibody;

(c) recovering the polyclonal antisera (i.e., a population of different antibodies) from the animal;

(d) optionally depleting the polyclonal antisera of antibodies not specific for a particular polypeptide conformer;

(e) isolating from the polyclonal antisera antibodies specific for a particular polypeptide conformer;

(f) determining the polypeptide sequence of the antibody specific for the polypeptide conformer;

(g) using the polypeptide sequence to design one or more first degenerate oligonucleotides comprising nucleotide sequences corresponding to polynucleotides encoding the N-terminal sequence of the mature (i.e., lacking a signal peptide) antibody heavy and light chains (or single chain antibody) specific for the particular polypeptide conformer;

(h) using a known database of antibody constant regions of the heavy and light chains (or single chain antibody) of the species in question to design one or more second oligonucleotides, which are complementary to nucleotide sequences encoding the constant region of the antibody heavy and light chains;

(i) using the oligonucleotides of (g) and (h) to amplify by polymerase chain reaction (PCR) polynucleotides encoding the antibody heavy and light chains (or single chain) specific for the particular polypeptide conformer; and

(j) expressing the antibody specific for a polypeptide conformer encoded by the polynucleotide sequence(s) identified in (i),

thereby producing an antibody specific for the polypeptide conformer. In effect, this method produces a monoclonal antibody (MAb) from a polyclonal antibody response. The MAb is specific for a particular polypeptide conformer and can be used to distinguish this particular conformer from other conformers.

In certain embodiments of the invention, the polypeptide composition enriched for the polypeptide conformer will be provided under non-denaturing conditions to avoid adversely affecting the three-dimensional structure of the polypeptides. In some cases, however, it may be desirable to provide the polypeptide composition under partially denaturing conditions.

In some embodiments of the invention, such polypeptide compositions may be produced using a polynucleotide encoding a signal peptide of a heterologous signal peptide, a modified signal peptide, a chimeric signal peptide, or combinations, thereof, to direct the folding and/or co- or post-translational processing of the polypeptides of the composition.

In some embodiments, the animal to which the conformer-comprising polypeptide composition is administered is selected from the group consisting of a rabbit, a mouse, a rat, a horse, a donkey, a hamster, a guinea pig, a bovine, an ovine, a primate (including a human), a camelid, a llama, or a related taxa. Other animals may be used, provided that they are amenable to antibody production. In some embodiments of the invention, the polyclonal antibodies produced by the animals are recovered from the animal by bleeding or exsanguinations, and RNA encoding the antibody is obtained by extraction from blood leukocytes, particularly B-cells.

In one embodiment of the invention, where two or more conformers can be enriched (e.g., by signal sequence domain swapping and expression) but only one conformer is of interest, the antibody specific for the polypeptide conformer of interest is isolated using affinity matrices for each polypeptide conformer. In one example, one polypeptide conformer (e.g., the conformer not of interest) is bound to a solid matrix and used to remove all antibodies common to all conformers as well as those specific for the conformer not of interest, with the flow-through (containing antibodies that did not bind to the conformer not of interest) applied to the second conformer column (to which the conformer of interest is immobilized) to affinity purify only the antibodies specific to that conformer of interest.

In another example, the conformer of interest is bound to insoluble particles and used to affinity purify the antibody specific for the polypeptide conformer, preferably after depleting the polyclonal antibody using the conformer not of interest, as described above. In yet another example, the polypeptide conformer of interest is used to immunoprecipitate the antibody specific for the conformer of interest.

In another example a mixture of conformers is separated by native gel electrophoresis, transferred to nitrocellulose or other membrane under native conditions and the membranes are cut into strips containing each conformer for use in affinity purifying antibodies that react to a given conformer either directly or after depletion of antibodies reactive to other conformers. Depletion may be accomplished as described above or by first incubating the polyclonal antibody with a membrane to which conformers that are not of interest have been immobilized.

In one embodiment of the invention, the polypeptide sequence of the antibody (or pool of antibodies) specific for a polypeptide conformer is determined by partial N-terminal amino acid residue sequencing of the heavy and light chains (or the single chain antibody, depending on the species) of the antibodies bound to a conformer of interest. Polypeptide sequence data so obtained is then used to design one or more degenerate oligonucleotides corresponding to polynucleotides predicted or known to encode the antibody specific for the polypeptide conformer. Such oligonucleotides may additionally comprise one or more restriction endonuclease sites or other sequences to facilitate identification and/or insertion into a heterologous polynucleotide, including but not limited to a plasmid, an expression vector, a cosmid, or an artificial chromosome. In one embodiment, the expression vector is a viral expression vector.

In one particular embodiment of the invention, the oligonucleotide or oligonucleotides are used to identify the coding sequence of the antibody specific for the polypeptide conformer from a library, such as a genomic library or a cDNA library or variations thereof. In another embodiment, the one or more oligonucleotides are used in a hybridization assay to identify polynucleotides encoding the antibody specific for the polypeptide conformer. Alternatively, the one or more oligonucleotides are used to amplify the coding sequence of the antibody specific for the polypeptide conformer using PCR methods along with mRNA isolated from the B-cells of the organism that produced the polyclonal antibody. If necessary, an additional oligonucleotide may be used, which need not be specific for a particular antibody but may rather correspond to the class or type of antibodies from which the polynucleotides encoding the antibody specific for the polypeptide conformer are being isolated.

In an embodiment of the invention, the polynucleotide sequences encoding the antibody specific for a polypeptide conformer are inserted into exogenous polynucleotides for expressing said antibody, thereby providing essentially unlimited quantities of the identical conformer-specific antibody. In a particular embodiment of the invention, the expression vector is a viral expression vector.

The invention also comprehends antibodies, fragments thereof, and derivatives thereof, produced by the methods described here. In one embodiment of the invention, for instance, the polynucleotides encoding the antibody specific for the polypeptide conformer are further modified, e.g., via sub-cloning and other molecular biology techniques, to express a modified version of the antibody specific for the polypeptide conformer, including but not limited to a single-chain antibody, a humanized antibody, a chimeric antibody, and an antibody fragment. One skilled in the art will recognize that it may be necessary to express more than one polypeptide, which will then be combined in appropriate stoichiometric amounts to produce functional antibodies, fragments thereof, or derivatives thereof.

The antibodies, fragments, thereof, or derivatives, thereof are useful for identifying particular polypeptide conformers in biological samples. Accordingly, the invention includes a method of using the antibodies, produced as described herein, to identify a polypeptide conformer in a sample comprising one or more conformers. In a preferred embodiment of the invention, the sample is provided under non-denaturing or partially denaturing conditions. In one example of such a method, the sample is obtained from a patient suffering from or at risk of suffering from a disease mediated by a polypeptide conformers. In a particular example, the disease is selected from the group consisting of a prion disease, a viral disease, a neurological disorder, and a cell proliferative disorder, such as cancer. Specific examples of such diseases include amyotrophic lateral sclerosis, prostate cancer, addictive disorders involving opiate or cannabinoid receptors, Ebola virus, HIV, HCV, influenza or any other viral infection infection, and transmissible cerebral amyloidosis or subacute (transmissible) encephalopathies, such as Creutzfeldt-Jakob disease, Gerstmann-Straussler-Scheinker syndrome, bovine spongiform encephalopathy, kuru, and fatal familial insomnia.

Yet another embodiment of the invention includes using an affinity-purified conformer specific antibody as provided by the invention to identify peripheral blood white blood cells or splenocytes that are specific for a desired response, e.g., using a fluorescent activated cell sorter (FACS). In one embodiment, such cells are select by the FACS and utilized for extraction of total RNA therefrom.

In yet another embodiment of the invention, the antibodies described, herein, are used to confer passive immunity to a patient suffering from a disease mediated by a polypeptide conformer, such methods comprising administering to the patient an antibody of the invention along with a suitable pharmaceutical carrier.

Yet another embodiment of the invention provides a method for immunizing a patient against a disease mediated by a particular polypeptide conformer, the method comprising administering to the patient an immunogenic amount of a polypeptide composition enriched for a polypeptide conformer. The polypeptide composition may be administered in one or more doses, optionally in the presence of a suitable pharmaceutical carrier. In a preferred embodiment of the invention, the patient's antibody titer of antibody specific for a particular polypeptide conformer, is measured at some time following administration of at least one dose.

The invention also includes a kit of parts for practicing the methods made possible by the instant disclosure. Such kits may comprise one or more conformer-specific antibodies, instructions for use of the antibody or antibodies in an assay to detect the presence of a particular polypeptide conformer in a sample, and instructions for interpreting results of the assay.

Such kits may also comprise instructions and reagents, including but not limited to particular polypeptide conformers and/or conformer-specific antibodies for use in detecting the presence of a particular conformer in sample suspected of comprising such a conformer.

### DETAILED DESCRIPTION OF THE INVENTION

Prior to describing embodiments of the invention, the following terms are defined. Terms that are not defined should be given their ordinary meaning in the relevant art:

Polypeptide conformer: A polypeptide, including associated carbohydrates, lipids, and other covalently or non-covalently attached substituents, having a particular three-dimensional structure, including but not limited to secondary structure, tertiary structure, and surface topology, that distinguishes the polypeptide from other polypeptides that presently have or formerly had the same amino acid residue sequence, i.e., primary structure. Polypeptide conformers may also be referred to as “conformers” as context allows. Polypeptide conformers may result from different co-translational and post-translational folding, different co-translational or post-translational modifications, or combinations, thereof. Polypeptide conformers that different in folding may be substrates for different post-translational modifications. As used, herein, the term “polypeptide conformers” or “conformers” includes associated carbohydrates, lipids, and co-and post-translational modifications, as well as necessary cofactors and other covalently and/or noncovalently attached substituents, including but not limited to additional polypeptides, nucleic acids, etc, which impart conformer-specific biochemical structural properties or biological functional properties and/or that are necessary to provide the polypeptide conformer in the particular three-dimensional folded shape to elicit an antibody response that is unique to the particular conformer.

Particular polypeptide conformers, particular conformers, conformers of interest, and similar expressions refer to conformers that are known or suspected to be associated with a particular disease state or condition or are otherwise of interest in view of the context in which they are discussed. In contrast, other conformers present in a polypeptide preparation are designated conformers not of interest, or the like.

Co-translational or post-translational modifications: Covalent and/or subtractions to a polypeptide that occur during or following its translation. Examples include but are not limited to glycosylation, phosphorylation, sulfonation/sulfation, amidation, acylation, acetylation, methylation, hydroxylation, ADP-ribosylation, carboxylation, adenylation, ubiquitination, famesylation, prenylation, metal addition, maturation, proteolytic cleavage, and other known but not listed additions and/or subtractions to a polypeptide.

Affinity purification of a polypeptide: Purification of a polypeptide using a reagent or combination of reagents that is/are specific for the polypeptide to be isolated, purified, or enriched for, including purification of a polypeptide conformer using a reagent, such as an antibody, antibody fragment, or antibody derivative, that is specific for the conformer to be isolated.

Specific for a polypeptide conformer: Selectively or preferentially binds to or interacts with a particular polypeptide conformer, including associated carbohydrates, lipids, and/or other covalently and/or non-covalently attached substituents, or fragment or derivatives, thereof, compared to other conformers of the same polypeptide, or fragments or derivatives, thereof.

Bound to a solid matrix or immobilized: Covalently or non-covalently attached to physical entities such as beads, resins, slides, plates, dishes, or wells, which facilitate manipulation of the bound substance.

Immunoprecipitate: To make insoluble or facilitate the isolation of a substance by attachment to an antibody or fragments or derivatives, thereof, which may optionally be attached to a solid matrix, including but not limited to protein A sepharose beads or equivalents, thereof.

Heterologous DNA sequence: A DNA sequence of a different origin, e.g., a different organism, animal, mammal, primate, or human, as context indicates.

Administering to an animal: Injecting or otherwise introducing into the body of an animal, in a single dose or in multiple doses.

Recovering the polyclonal antibodyfrom an animal: Harvesting, sampling, or otherwise collecting biological materials comprising a polyclonal antibody raised to a subject antigen, such as a polypeptide conformer or biological composition comprising a conformer. Typical methods include exsanguinations and bleeding.

Degenerate oligonucleotides: Oligonucleotides having a sequence predicted to encode a subject polypeptide or fragment thereof. Such oligonucleotides may account for codon usage in a particular species and may include additional sequences, for example, to facilitate cloning, identification, or further amplification,

Suitable pharmaceutical carrier: Examples of suitable carriers (excipients) include buffers, solvents, coatings, lubricants, stabilizers, etc. Examples include but are not limited to lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propyl hydroxy-benzoates; sweetening agents; and flavoring agents, as well as enteric layers or coatings. Other suitable carriers are found in, e.g., REMINGTON'S PHARMACEUTICAL SCIENCES, Mace Publishing Company, Philadelphia Pa. (1985 and later editions).

Heterologous signal peptide: A signal peptide not associated in nature with a subject polypeptide. Heterologous signal peptides may be from a different polypeptide, from a different species, strain, or clinical isolate of an organism, or both. Heterologous signal peptides also include synthetic signal peptides.

Modified signal peptide: A signal peptide differing from a subject signal polypeptide at one or more amino acid residues, including residues in the N-terminal domain, hydrophobic domain, and/or C-terminal domain of the signal peptide.

Chimeric signal peptide: A signal peptide comprising amino acid residue sequences from more than one signal peptide.

Producing a polypeptide composition enriched for a polypeptide conformer: Producing or causing to be produced a polypeptide composition having a higher proportion of a subject polypeptide conformer than found in a corresponding natural biological source, such as tissues, cells, and serum.

Animal suspected of having or at risk of contracting a disease mediated by a polypeptide conformer: An animal, including but not limited to a human or non-human patient, with one or more symptoms of a polypeptide conformer-mediated, a familial history of a polypeptide conformer-mediated disease, exposure or contact with objects, biological materials, animals, or people known and/or suspected of harboring infectious agents associated with a conformer-mediated disease, presence in a location endemic for a conformer-mediated disease.

Electrophoretic mobility: The movement of a protein polypeptide, or fragment or derivative thereof, including a polypeptide complex, having a plurality of non-covalently associated polypeptides, in an electrical field.

Native gel electrophoresis: A form of gel electrophoresis that does not include denaturing agents such as sodium dodecyl sulfate (SDS), guanidinium-HCl, urea, 2-mercaptoethanol (2-ME or β-ME), and the like, or is substantially free of such agents

Denaturing agent: A chemical or treatment that modifies the quaternary associations or two or three-dimensional structure of a polypeptide. Examples include but are not limited to sodium dodecyl sulfate (SDS), guanidinium-HCl, urea, and 2-mercaptoethanol (2-ME or P-ME), as well as other chaotropic agents.

Immunological reactivity: The ability of a polypeptide, or fragments or derivatives, thereof, to be detected by an antibody (or mixture of antibodies) under a particular set of conditions. Antibodies react with epitopes of proteins that may be linear or discontinuous. The ability of an antibody to detect or react with a protein is reflected in the affinity and avidity of binding.

### Description of the Preferred Embodiments

Applicants have discovered that a growing number of polypeptides exist in different three-dimensional conformations, called “conformers” here, which have different biological functions despite having the same or substantially the same primary polypeptide sequences. The existence of different polypeptide conformers now has been shown in a number of disease states, including prion diseases, viral infection, prostate cancer, and amyotrophic lateral sclerosis, which are discussed below.

Prion Diseases:

Prion diseases are caused by the accumulation of a specific conformer of prion protein (PrP), which differs in its folded structure, i.e., its conformation, from other forms of the polypeptide. The form of the PrP found in the normal adult brain is termed ^{Sec}PrP (or PrPSc). However, ^{Ctm}PrP, a form of the polypeptide that is normally degraded in healthy individuals, accumulates in the brains of individuals suffering from prion disease, inducing apoptosis and resulting in neurodegeneration. Specific machinery of the endoplasmic reticulum (ER) is involved in regulating whether PrP is made in the Sec- or Ctm forms (Hegde, R. S. et al. (1998) Molecular Cell 2:85-89; Fons, R. D. et al. (2003) J. Cell. Biol. 160:529-39; and Hegde, R. S. et al. (1998) Science 279: 827-34).

Ebola Virus:

Ebola virus subspecies have been identified and named according to the area of their emergence. Different subspecies of ebola virus exhibit differential pathogenicity in humans. Infection by some subspecies, such as ZEBOV (a subspecies isolated in Zaire) but not others, such as SEBOV (Sudan), appears to be enhanced by interaction of the viral glycoprotein with C-type lectins present on the surface of host cells. Experimental evidence suggests that the signal peptides of the respective glycoproteins determines the extent to which they are folded and/or glycosylated into conformers that interact with the cellular lectins, contributing to differences in Ebola virus pathogenicity.

Prostate Cancer:

Prostatic acid phosphatase (pacp) is a polypeptide associated with prostate cancer. The presence of elevated levels of pacp in the blood of human males is still used as an indicator of prostate cancer; although, pacp is observed in men with non-cancerous prostate disease and is not always present in cases of prostate cancer.

Pacp is found in both intracellular and secretory forms, which are identical in a sequence and modifications (Lin, M. F. et al. (1998) J. Biol. Chem. 273:5939-47). Loss of the intracellular form correlates with development of prostate cancer; and conversion of pacp expression from an intracellular to a secretory form is associated with progression of prostate cancer from an androgen-sensitive to an androgen-resistant phase (Yeh, S. et al. (1999) Proc. Nat'l. Acad. Sci. U.S.A. 96:5458-63).

Amyotrophic Lateral Sclerosis:

Amyotrophic lateral sclerosis (ALS), also called Lou Gehrig's disease, is a progressive, fatal neurological disease affecting as many as 40,000 Americans with 5,000 new cases occurring in the United States each year. A familial form of ALS (FALS) accounts for 10% of all cases, about 20% of which are associated with a particular form of the copper-zinc superoxide dismutase-1 enzyme (SOD 1), a 32 kD homodimeric anti-oxidant. Rosen et al. (1993) Nature 363:59-62.

Experimental evidence shows that the distribution of SOD1 conformers is different in spinal chord tissue obtained from individuals with ALS, compared to healthy individuals. Specifically, cell extracts prepared from spinal chord tissue obtained from patients with either familial- or sporadic ALS have a distinct conformer of SOD1 that can be readily distinguished by chemical modification with NHS biotin followed by polyacrylamide gel electrophoresis in sodium dodecyl sulfate and western blotting. These results suggest that the absence, rather than the presence, of a particular SOD1 conformer is associated with the disease state.

Prion diseases, prostate cancer, ALS, and Ebola virus infection are likely only a small fraction of the diseases and disorders that are likely to be associated with particular polypeptide conformers. Taken together, the above observations suggest that polypeptide conformers are implicated in a variety of different diseases, defying the traditional notion of “one gene—one protein,” and complicating the process of identifying polypeptides involved with diseases.

Conformer association with disease is suggested when a disease state is not associated with a gene mutation that affects the amino acid sequence of a polypeptide known to be associated with the disease or that affects the expression levels or turnover of such a polypeptide. In essence, conformer association with disease is implicated when traditional acquired or hereditary genetic causes for a disease have been ruled out, yet there exists a defect in cell function, which is not merely a consequence of environmental conditions.

In such cases, it is possible that multiple polypeptide conformers of a polypeptide are being produced in a cell, and such conformers may have different biological functions (including different levels of biological function). The recognition that such polypeptide conformers exist and that different conformers of the same polypeptide may have different biological functions, is an important aspect of the instant invention.

Without being limited to a theory, the invention is based on the observation that polypeptides that were once believed to exist in a relatively homogenous folded form often exist as a population of different folded forms, herein referred to as “polypeptide conformers.” A population of polypeptides having the same amino acid residue sequence may exist in several thermodynamically stable conformations, which have different biological activities (or different levels of biological activity), and which play different roles in cell signaling and/or metabolism.

While it has been recognized that “misfolded” polypeptide may be produced in cells, the prevailing wisdom has been that such misfolded polypeptides generally are non-functional and are targeted for destruction by the cellular proteosome apparatus. In contrast, the present invention is predicated on recent evidence that that many if not most proteins are produced as different or alternative conformers. The different polypeptide conformers may actually have different biological activities, with particular conformers being exclusively or predominantly associated with particular cell types, particular windows of time in development, particular metabolic states or particular disease states or points in the natural history of disease, all, in principle, in a genetically and environmentally modifiable fashion.

A major obstacle in the identification of polypeptide conformers is the lack of suitable reagents for their detection. Up until this point, identifying different protein conformers was virtually impossible because the tools were not available to distinguish between different conformers. Moreover, since polypeptides are frequently analyzed under denaturing conditions (e.g., in the presence of SDS, 2-ME, urea, and the like), the art has largely ignored the existence of different protein conformers. In addition, since many polyclonal antibodies are raised against denatured polypeptides or small synthetic peptides corresponding to portions of polypeptides of interest, they usually lack the ability to distinguish between different polypeptide conformers.

While it may be possible to use hybridoma technology to produce monoclonal antibodies specific for a particular polypeptide conformer, this approach is plagued by several difficulties. For example, the polypeptide preparation used as the antigen to inoculate the animal may comprise only a small fraction of the conformer of interest, skewing the production of polyclonal antibodies toward those that recognize conformers that are not of interest, with the production of few antibodies that recognize the conformer of interest (i.e., antibodies of interest). Furthermore, isolating the antibodies that are specific for the conformers of interest, if present, requires the use of hybridoma technology, which is expensive, labor intensive, and results in the production of a cell line, which must be maintained in culture, when not frozen in liquid nitrogen, which is subject to contamination, destruction in storage due to interruption of liquid nitrogen service, and genetic variation over time. Moreover, even under the best of circumstances, hybridoma technology, in the absence of further genetic manipulation, does not yield isolated polynucleotides or polynucleotide sequence data that would be useful for generating humanized, single chain, or other modified antibodies, or expressing such antibodies in a heterologous organism.

Antibodies specific for particular conformers of a polypeptide, particularly conformers associated with a disease state, would be powerful reagents for diagnosing and monitoring the progress of disease, and in developing therapies for treating conformer-associated disease. However, as discussed above, existing polyclonal and monoclonal antibody technologies are not readily amenable to producing conformer-specific antibodies.

The instant invention provides an elegant solution to this problem, which combines some of the most desirable features of polyclonal antibody production with those of monoclonal antibody production. According to the methods of the invention, laboratory animals, such as rabbits, are used to generate high-affinity polyclonal antibodies to particular immunogen preparations, which comprise conformers of interest. In most embodiments, the resulting polyclonal antibodies are then subjected to biochemical fractionation to isolate conformer of interest-specific antibodies, which are present in the polyclonal antibody population. Having identified the conformer-specific antibodies in the population, the amino acid residue sequence of such antibody heavy and light chains is determined and used to design reagents for cloning polynucleotides encoding conformer-specific antibodies, which may then be further manipulated and used to express conformer-specific antibodies.

The instant invention solves these problems by identifying a polynucleotide encoding a conformer-specific antibody, which can be further manipulated to allow the expression of a variety of different types of antibodies, including humanized, single-chain, or other types of antibodies, and can be stored indefinitely in solution or as a precipitate.

The invention provides methods for producing conformer-specific antibodies, the conformer-specific antibodies produced by such methods, and methods of using the conformer-specific antibodies for diagnosing and treating diseases associated with polypeptide conformers. The invention further provides methods for use of the polypeptides and antibodies in identifying and isolating drug targets.

Obtaining Polypeptide Compositions for Immunizing Animals:

Another important feature of the invention is the ability to initially obtain the conformer of interest from natural biological sources or to be able to make or produce the conformer of interest, in vivo or in vitro. The availability of polypeptide preparations that are enriched for conformers of interest allows the ultimate production of the conformer-specific antibodies of the invention, which can then be used in subsequent antibody-based identification and/or detection of the conformer of interest.

While a pure form of the conformer is clearly desirable, polypeptide preparations enriched for the conformer of interest are more likely to be available. Accordingly, as used herein, a polypeptide preparation enriched for a conformer of interest includes such pure preparations, with the understanding that they are unlikely to be readily available.

Polypeptide preparations enriched for a particular conformer may be prepared using polypeptide separation methods that distinguish between different conformers of the same of substantially the same polypeptide based on surface topology (including surface charge), shape, or other features that would distinguish polypeptides that differed in three-dimensional structure but not necessarily in amino acid sequence. Examples of such methods include but are not limited to ion exchange chromatography, native polyacrylamide gel electrophoresis (native PAGE), two-dimensional (2-D) gel electrophoresis, sedimentation methods, affinity chromatography, and combinations and/or variations, thereof.

Conformers of interest may also be obtained by directing the folding and/or co- and post-translationally modification polypeptides such that cells (or a cell-free translation system) are more likely to produce a conformer of interest, as opposed to conformers that are not on interest. One way of directing the folding of a polypeptide is accomplished by substituting or “swapping” the naturally occurring signal peptide of a polypeptide (where applicable) with a heterologous signal peptide that directs such desired folding. The heterologous signal peptide may be from the same organism, a different organism, or even a different species. The signal peptide may be chimeric, mutated, substituted, truncated, or entirely synthetic (i.e., designed or engineered using the knowledge available in the art). The signal peptide may be “swapped” in its entirety or portions of the signal peptide may be “swapped.” In some embodiments, the N and/or H domains of the signal peptide are swapped. In a particular embodiment, the signal peptide of ZEBOV is used to direct the production of pathogenic forms of Ebola virus glycoprotein from non-pathogenic subspecies of Ebola virus.

In some cases, the signal peptide that is most likely to direct the production of the conformer of interest will be known or readily determined. In other cases, it may be necessary to try several signal peptides to determine which one directs the production of the greatest amount of the conformer of interest. This procedure is readily accomplished using standard molecular biology techniques, particularly cloning, plasmid manipulation, and polypeptide expression in host cells and/or cell-free systems.

Signal sequence “swapping” can also be combined with, or replaced by a variety of biochemical separation techniques, thereby providing two methods for enriching a polypeptide preparation for conformers of interest.

In one embodiment of the invention, such separation techniques and methods are employed under non-denaturing conditions to separate particular fractions, isolate, or enrich for polypeptide conformers present in a composition comprising a plurality of conformers.

In another embodiment of the invention, partially denaturing conditions are utilized to exploit differences in the stability of different conformers against chemical, thermal, oxidative, and/or reductive denaturants. This embodiment it preferable where different conformers in a population are denatured by different concentrations of a denaturing agent and/or denature at different rates upon exposure to a particular denaturing agent, and represents another means of distinguishing between different conformers. These populations of conformers, in selectively denaturing conditions, are now subjected to the methods of enrichment described above and known in the art.

In one particular embodiment of the invention, the conformers of interest are more stable than the conformers that are not of interest. In another embodiment, the conformers of interest are less stable than the other conformers. In either case, it may desirable or necessary to refold the partially denatured conformers following enrichment. Since renaturation may result in the formation of misfolded polypeptide, or even conformers that are not of interest, one skilled in the art will generally recognize the advantages of avoiding denaturing conditions when dealing with particular polypeptide conformers.

Different chromatographic fractions can then be assayed for biological activity prior to use as a source of antigen to inoculate animals. As discussed, above, particular conformers are associated with particular biochemical characteristics, such as surface topology and particular biological activities, such as mediating a disease state. Therefore, using appropriate biochemical or biological assays, it is possible to identify chromatographic fractions enriched for a conformer of interest, and using such enriched fractions as a source of antigen to inoculate an animal.

Where no biological or biochemical assay exists to distinguish conformers, it may be desirable to raise antibodies against a series of chromatographic fractions, using multiple animals, then later sort out the specificities of such antibodies.

While any amount of enrichment may increase the potential to obtain the desired conformer-specific antibodies, the chromatographic methods should, ideally, produce a polypeptide preparation (i.e., pooled chromatographic fraction) that is at least 5-10% conformer of interest. Preferably, the polypeptide preparation is at least 10-20% conformer of interest or even 20-30% conformer of interest. Ideally, the preparation is greater than 30% conformer of interest.

Methods for Producing Antibodies

Polyclonal antibodies can be raised against the above-described polypeptide preparations by various procedures that are well known in the art. For example, a polypeptide preparation enriched for a conformer of interest can be administered to host animals including, but not limited to, rabbits, guinea pigs, mice, rats, sheep, goats, cows, horses, donkeys, dogs, primates, and the like. The use of rabbits is exemplified, herein; however, the methods of the invention are applicable to a wide variety of animals without departing from the invention. Of particular interest are animals such as camels, which produce single chain antibodies, which are easier to clone and express than antibodies comprised of heavy and light chains.

Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Adjuvants are well known in the art and not part of the invention.

Antibody production can be monitored using methods known in the art, which typically involves incubating immune and pre-immune serum with the polypeptide conformer of interest and determining whether complexes are formed. ELISA, immunoblot, and radioimmunoassay are known in the art and one skilled in the art will know how to monitor the production of antibodies following inoculation of an animal.

In some embodiments, the animal used to provide the antibodies is immunized with known or suspected conformers, with peptides, or even completely denatured recombinant protein, any combination of these, or all three. A combinational approach is useful, as one method of immunization may generate a response missed by the others.

Once you have a desired titer is achieved, e.g., the response is at maximal achievable titer, then cloning of the desired antibody or antibodies is performed as described herein. In one embodiment, the cloning is performed by purifying the desired conformer, generating B-cell total RNA, and amplifying the full length clones (e.g., using PCR) and selecting the desired heavy-light chain combination by expression in transfected cells (e.g., CHO cells).

Identification of Conformer-Specific Antibodies in the Polyclonal Antibody:

If the polypeptide preparation enriched for a conformer of interest is homogenous, or substantially homogenous for the conformer of interest, the polyclonal antibody may be useful as a reagent to detect the conformer without further manipulation. However, in most cases, such polyclonal antibodies may comprises a mixture of conformer-specific antibodies and benefit from further manipulation. Thus, one skilled in the art will recognize that crude polyclonal antibodies are usually an intermediate product on the way to producing a conformer specific antibody.

Once a polyclonal antibody is obtained, it is harvested from the animal, typically by bleeding or exsanguination, and used for subsequent procedures, which should be performed under non-denaturing conditions. For example, the IgG fraction may be isolated and incubated in the presence of the polypeptide preparation enriched for the conformer of interest, optionally, in parallel with incubation in the presence of a polypeptide preparation that is not enriched with the polypeptide of interest, and along with polypeptide preparations in the absence of the IgG fraction. The resulting antigen-antibody complexes are then resolved on a non-denaturing gel. Antibody-antigen complexes will show altered migration or a “gel-shift,” compared to the results obtained using either the antigen or antibody, alone.

The polypeptides present in the retarded or “shifted” bands of the non-denaturing gel can be excised and the polypeptides eluted and fractionated into H and L chains, preferably under denaturing conditions. The fractions then are subjected to amino acid sequence analysis, which will reveal the presence of a single IgG or a mixed population of several distinct IgGs. The latter situation will be evident from the appearance of multiple chromatographic “peaks” or “signals” at the same amino acid position, depending on the particular method of sequence analysis employed.

Alternatively, it may be desirable to use affinity chromatography to isolate antibodies that bind to the conformers of interest, ideally after first depleting the polyclonal antibody population of antibodies that bind to conformers that are not of interest or bind to epitopes present or exposed on multiple conformers. One way to achieve this depletion is to subject the polyclonal antibody preparation to a series of at least two chromatographic purification steps, wherein the chromatographic steps involve different immobilized conformers. For example, if a polyclonal antibody is first passed over a column onto which conformer “A” has been immobilized (bound) and the eluate (flow through) from this column is then passed over a column to which conformer “B” is immobilized, the antibodies that bind to the second column will be specific for conformer “B” and depleted for antibodies that bind to “A” or both “A” and “B”. In this manner, antibodies that recognize the conformer of interest (“B” in the above example) are isolated from antibodies that recognize the conformer of interest as well as conformers that are not of interest.

While there are numerous variations of the above-described depletion scheme, the important feature is that the conformer of interest is not present in the first chromatographic step (i.e., not bound to the column), which is used to deplete the polyclonal antibody of unwanted antibody sub-species. However, the conformer of interest is present in the second chromatographic step, which is used to bind and isolate antibodies specific for the conformer of interest.

One notable variation of the above depletion and enrichment scheme is to add conformers of interest directly to the eluate from the first column, optionally concentrate the resulting antibody-conformer of interest complexes (e.g., by filtration, centrifugation, precipitation, dialysis, etc.), then resolve the complexes under nondenaturing conditions, e.g., using a native polyacrylamide gel. The complexes are then eluted or excised from the gel, optionally transferred to a membrane or other suitable carrier, and finally subjected to amino acid sequence analysis.

One skilled in the art will recognize that multiple depletion steps may be necessary to remove all unwanted antibodies from the polyclonal population. At the very least, it may be desirable to pass the polyclonal antibody preparation over the first column (to which conformers not of interest are bound) multiple times or to use multiple columns in series. This process increases the efficiency of depletion.

Another convenient variation of the above approach is to resolve a polypeptide preparation comprising a conformer of interest on a native gel, transfer the resolved polypeptides onto a membrane (maintaining denaturing conditions) then use the resulting membrane to bind antibodies present in a solution (e.g., serum). The serum may first be exposed to a membrane that comprises conformers that are not of interest (i.e., conformer “A”) to deplete the serum of unwanted antibodies. Following depletion, a membrane comprising conformer “B” is used to bind the antibodies specific for the conformer of interest.

Similarly, the serum can be divided and incubated in the presence of membranes comprising either conformer “A” or conformer “B”. Bound antibodies are then eluted from the membrane (as free antibodies or fragments in solution) and depleted of cross-reacting antibodies using a membrane or equivalent, thereof, comprising the other conformer (“A” or “B”). The antibodies remaining in solution are specific for conformer “A” or “B”, while the bound antibodies cross-react with conformers “A” and “B.”

An advantage of the membrane method is that it obviates the need to identify gel shift conditions, which although routine in the art, can nonetheless be tedious. As was the case above, variations of this method will be apparent in view of the disclosure and it may be desirable to combine the “membrane methods” with other methodologies described, above, such as immunoprecipitation.

These depletion steps may be combined with other methods of antibody purification described above and below, or known in the art. One skilled in the art will also recognize that antibody fragments, e.g., Fab fragments, may be used rather than intact antibodies. Additional modifications will be apparent in view of this disclosure.

Following isolation of the antibodies specific for the conformer of interest, N-terminal sequencing, such as by Edman degradation, or derivative methods, are employed to determine the identity of the N-terminal amino acid residues of the mature heavy and light chains of the antibody (or the single chain, depending on the species). Since the antigen-binding site of an antibody is generally proximal to the N-termini of the individual polypeptide chains, the identification of polypeptide sequences responsible for conformer-specific antibody binding is readily determined by N-terminal sequencing. Sufficient sequencing is performed to allow the design of oligonucleotide primers, as described, below. Generally, the identity of the N-terminal 10-25, preferably 12-20, and most preferably 15-18 amino acid residues of each chain should be determined.

Designing Oligonucleotide Primers Based on the Polypeptide Sequence Data:

Knowing the polypeptide sequence (or average sequence with known positions of heterogeneity) of conformer-specific antibodies, in view of the standard genetic code and the frequency of codon usage in the organism used to produce the polyclonal antibody, allows the design of degenerate oligonucleotide primers for use in identifying all or portions of the polynucleotide coding sequences of the conformer-specific antibodies.

Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; and Ike et al. (1983) Nucleic Acid Res. 11:477, and the thermodynamic criteria (e.g., in terms of hybridization/annealing temperatures) for oligonucleotide primer selection are known in the art.

Multiple oligonucleotides may be designed to maximize the chance that at least one oligonucleotides will hybridize to the target polynucleotide sequences in the library that encode the conformer-specific antibody. Each of these oligonucleotides may represent the coding sequence of the conformer-specific antibody but may differ in polynucleotide sequences to account for codon variation in the third position and first and second positions, where applicable (e.g., for the codons of serine, leucine, and arginine). In this manner, the chances of obtaining oligonucleotide primers that hybridize to target polynucleotide sequences is increased.

One skilled in the art will recognize that polypeptide sequencing of a mixed population of polypeptides produces an “average” sequence, rather than multiple discrete sequences. Thus, where polypeptide sequence data reveals the presence of multiple antibodies that bind to a polypeptide conformer, it may be desirable to produce a series of oligonucleotides, corresponding to the coding sequences of different antibodies that potentially exist in the population, based on different permutations of the amino acid residues determined to be heterogenous in the population of H and/or L chains.

These oligonucleotides may be used in a PCR reaction to amplify polynucleotides, e.g, or as “probes” or primers encoding conformer-specific antibodies from a library or used according to hybridization methods as to identify such polynucleotide in a library.

Methods of making and using libraries are well-known in the art. Reagents for their production and screening are commercially available. Ideally, the libraries are prepared from appropriate biological samples obtained from the same animal that was used for the production of the polyclonal antibody, in order that the target antibody coding sequence be represented in the library. Methods for screening libraries are known in the art and exemplified in U.S. Pat. Nos. 6,319,690 and 6,436,677, each of which is incorporated by reference in its entirety. The use of cDNA libraries is preferred, e.g., because of the absence of introns; however, since antibody diversity results from DNA rearrangements, genomic libraries will also comprise the particular coding sequences of the conformer-specific antibodies, and may also be used.

In the case of identifying the polynucleotides by PCR or other primer-extension techniques, only a single oligonucleotide need hybridize to the portion of the target polynucleotide sequence that encodes the portion of the antibody that is specific for the conformer of interest. For example, in the case of PCR methods, a second primer may be used, which hybridizes to a conserved region of the antibody coding sequence. These second primers may be species specific but may also be specific for a particular class of antibody, e.g., IgG, IgM, IgA, IgE, IgD, IgY, and the like, or even subclasses of antibodies, some of which are described or referenced, herein. As in many methods involving cases PCR or primer extension, it may be desirable to include heterologous nucleotide sequences in the oligonucleotides, particularly in their 5′ ends, e.g., to facilitate further amplification, to provide sites for restriction endonucleases, or otherwise facilitate the manipulation of the resulting polynucleotide product ultimately obtained using the oligonucleotide primers described, herein. Such primers may also be conjugated to non-nucleoside components, such as peptides, fluorescent groups, quenching groups, solid particles, and the like, to permit subsequent identification and/or manipulation. One skilled in the art will recognize that oligonucleotides may be designed to incorporate a number of additional features without departing from the scope of the invention.

It is also understood that in some cases, it may be necessary to iteratively reduce the annealing temperature in PCR reactions, or decrease the stringency of hybridizations conditions, until polynucleotide are identified using the degenerate primers.

Alternatively, provided that sufficient sequence data is obtained using N-terminal or other forms of polypeptide sequencing, it may be possible to construct all or a part of an antibody coding sequence using synthetic oligonucleotides, inserted into a convenient antibody expression vector. In this manner, the need to isolate polynucleotides from a library is avoided.

The amount of the antibody coding sequence that can be made using synthetic oligonucleotides is not limited to the region that is sequenced. As described, above, antibodies may be constructed entirely from synthetic oligonucleotides, in which case the practitioner has broad discretion in determining what type of antibody to produce, e.g., a humanized antibody, a single chain, antibody, etc. As this point, the combination of different antibody products that can produced according to the invention is bound only by the type of antibodies that are known in the art, and for which isolated polynucleotide sequences are available or can be obtained or synthesized.

As another alternative to screening a library, polynucleotides encoding conformer-specific antibodies can be identified using an antibody phage display library. Kits for generating and screening phage display libraries are commercially available (e.g., PHARMACIA Catalog No. 27-9400-01 and STRATAGENE Catalog No. 240612).

Additionally, examples of methods and reagents useful for generating and screening antibody display library can be found in, e.g., U.S. Pat. No. 5,223,409 (Ladner et al.); WO 92/18619 (Kang et al.); WO 91/17271 (Dower et al.); WO 92/20791 (Winter et al.); WO 92/15679 (Markland et al.); WO 93/01288 (Breitling et al.); WO 92/01047 (McCafferty et al.); WO 92/09690 (Garrard et al.); WO 90/02809 (Ladner et al.); Fuchs et al. (1991) Bio/Technology 9:1369-72; Hay et al. (1992) Hum. Antibod. Hybridomas 3:81-85; Huse et al. (1989) Science 246:1275-81; Griffiths et al. (1993) EMBO J. 12:725-34; Hawkins et al. (1992) J. Mol. Biol. 226:889-96; Clackson et al. (1991) Nature 352:624-628; Gram et al. (1992) Proc. Natl. Acad. Sci. USA 89:3576-3580; Garrard et al. (1991) Bio/Technology 9:1373-1377; Hoogenboom et al. (1991) Nucleic Acids Res. 19:4133-37; and Barbas et al. (1991) Proc. Natl. Acad. Sci. U.S.A. 88:7978-82.

In yet another embodiment of the invention, polynucleotide sequences encoding the heavy and light chains of the antibodies that bind the conformers of interest are amplified using mRNA isolated from lymphocytes of the animal used to produce the polyclonal antibody starting material. The use of poly(A) RNA is preferred although not strictly necessary. It may also be desirable to enrich the lymphocytes for B-cells, which produce antibodies. Methods for enriching a cell population for B-cells, or subpopulations, thereof, typically involve fluorescence-activated cell sorting (FACS) or chromatographic purification using known B-cell markers. Such methods are well-known in the art and do not constitute part of the invention.

Expression of Antibody-Encoding Polynucleotides:

A variety of host-expression vector systems may be utilized to express antibody molecules of the invention. Such host-expression systems include those that express H- or L-chains for subsequent isolation and combination to yield functional antibodies as well as expression systems that produce mature, partially or completely co- and/or post-translationally-modified antibodies. These include but are not limited to bacteria, yeast, insect cells, plant cells, or animal cells, including mammalian cells.

One skilled in the art will recognize that there is no shortage of bacterial, yeast, insect, or expressions, which are commercially available or described in the literature. It has also become a routine matter to design and construct expression vectors comprising particular enhancers, promoters, polylinkers, introns, polyadenylation signals, initiation sequences, termination signals, selectable markers, and other features to control expression and/or facilitate recovery and isolation of the expressed polypeptide. Expression vectors are not part of the invention and will not be described further.

A host cell strain may be chosen which modulates the expression of the inserted sequences or modifies and/or processes the polypeptide content in a specific fashion. Such modifications (e.g., glycosylation) and processing (e.g., cleavage) may be important for the particular biochemical structure or biological function of the polypeptide conformer of interest. Different host cells have characteristic and specific mechanisms for the co- and post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to COS, CHO, BHK, HeLa, COS, MDCK, 293, 3T3, W138, and various lymphocyte derived cells, including B-cell-derived cells.

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot (1986) Nature 322:52; Kohler (1980) Proc. Nat'l. Acad. Sci. U.S.A. 77:2197). The coding sequences for the heavy and light chains may comprise cDNA or genomic DNA.

Following expression and purification, and if necessary, mixing heavy and light chain preparations, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of antibodies or other polypeptides. In addition, the antibodies of the present invention or fragments thereof can be fused to heterologous polypeptide sequences described herein or otherwise known in the art, to facilitate purification.

For long-term, high-yield production of recombinant proteins, stable expression may be preferred.

One exemplary sequence of operations using the oligonucleotides prepared according to the present invention to produce antibodies:
1) Immunize the animal (e.g., intraperitoneally) approximately 1-3 boosts over 1 week.
2) Collect about 10 to about 50 mls of heparinized peripheral blood approximately 1 week post boost.
3) Collect the buffy coat (white blood cells) from the heparinized peripheral blood.
4) Extract total RNA by any of several methods, including phenol choloroform isoamylalcohol extraction, followed by LiCl precipitation and ethanol precipitation.
5) Use the oligonucleotides designed above to do rtPCR from this total RNA. The oligonucleotides preferably encode a multicloning site to allow the PCR product to be subcloned into a suitable expression vector.
6) Transfect a suitable cell line (e.g., CHO cells) with plasmid DNA extracted from individual colonies or pools of colonies for heavy chain and for light chain antibody fragments.
7) Collect media from transfected cells and screen for conformer specific antibody.
8) Take positive clones from the primary screen and validate them with secondary screens.
   Types of Antibodies That Can be Expressed as Part of the Invention:

Once the polypeptide sequence that makes an antibody specific for a particular conformer is known, e.g., via N-terminal sequencing, and methods, such as those described, herein, are used to identify coding sequences for that particular region of the antibody, one skilled in the art will recognize that a wide range of antibody products may be constructed using standard molecular biology techniques.

Accordingly, antibodies of the invention including, but are not limited to monoclonal intact antibodies, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. Methods for producing some of these type of antibodies are described in, e.g., International Application No. PCT/US86/02269 (Robinson et al.); European Patent Applications 184,187 (Akira, et al.); 171,496 (Taniguchi); 173,494 (Morrison et al.); 125,023 (Cabilly et al.); WO 86/01533 (Neuberger et al.); U.S. Pat. No. 4,816,567 (Cabilly et al.) and U.S. Pat. No 5,225,539 (Winter); Better et al. (1988) Science 240:1041-43; Liu et al. (1987) Proc. Natl. Acad. Sci. U.S.A. 84:3439-43; Liu et al. (1987) J. Immunol. 139:3521-26; Sun et al. (1987) Proc. Natl. Acad Sci. U.S.A. 84:214-18; Nishimura et al. (1987) Cancer Res. 47:999-1005; Wood et al. (1985) Nature 314:446-49; Shaw et al. (1988) J. Natl. Cancer Inst. 80:1553-59); Morrison (1985) Science 229:1202-07; Oi et al. (1986) BioTechniques 4:214; Jones et al. (1986) Nature 321:552-25; Verhoeyen et al. (1988) Science 239:1534; and Beidler et al. (1988) J. Immunol. 141:4053-60, which are incorporated by reference.

The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. In a preferred embodiment, the immunoglobulin is an IgG1 isotype. In another preferred embodiment, the immunoglobulin is an IgG2 isotype. In another preferred embodiment, the immunoglobulin is an IgG4 isotype. Immunoglobulins may have both a heavy and light chain. An array of IgG, IgE, IgM, IgD, IgA, and IgY heavy chains may be paired with a light chain of the κ- or λ forms.

The antibodies may be human antigen-binding antibody fragments, including but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of other regions of the antibody.

The antibodies of the present invention may be monospecific, bispecific, or of greater multispecificity. However, since the antibodies preferentially recognize particular conformers of a polypeptide, it is most likely that the antibodies will be specific for only a few epitopes that are unique or more accessible in the conformers of interest.

Such specificity may be the result of folding, glycosylation, and/or other modification that are specific or preferred for the conformer of interest, some of which are identified, herein. In particular embodiments the polypeptide conformer of interest is produced because of the particular signal peptide associated with the polypeptide.

Examples of particular embodiments of the invention include, but are not limited to, antibodies that recognize conformers of Ebola virus glycoprotein that are associated with pathogenicity, conformers of SOD1 that are associated with disease, conformers of PAcP that are associated with prostate cancer or worsening, thereof, conformers of cannabinoid receptor CB1 associated with alcoholism or drug addiction, conformers of CFTR associated with lung disease, and ^{Ctm}PrP, which is associated with prion disease.

It may also be desirable to produce, using the methods described herein and/or apparent in view of the disclosure, antibodies that are specific for polypeptide conformers that are not associated with a disease state. One skilled in the art will recognize that such antibodies may be used for many of the same purposes as antibodies specific to disease-associated conformers, bearing in mind the important differences in interpreting the results of assays using these antibodies.

In a most preferred embodiment of the invention, antibodies specific for each polypeptide conformer are prepared, thereby allowing the identification of each conformer species present in a biological sample, providing the most thorough means for identifying different conformers in a population of different conformers.

Modification of Conformer-Specific Antibodies:

The antibodies of the invention include derivatives that are modified, i.e, by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, PEGylation, phosphorylation, amidation, derivatization by known protecting and blocking groups, proteolytic cleavage, linkage to a cellular ligand or other protein, etc. Any of numerous chemical modifications may be carried out by known techniques

Utility:

One skilled in the art will recognize that the invention has many utilities, only some of which are described, herein.

One useful feature of the invention is that it provides essentially “monoclonal” antibodies without the need to employ hybridoma technology. By using as antigens polypeptide preparations that are enriched for a particular conformer, in combination with biochemical methods to isolate conformer-specific polyclonal antibodies (e.g., using the native “gel shift” methods described, herein), followed by polypeptide sequence analysis, the invention provides a rapid means of isolating and cloning polynucleotides encoding conformer-specific antibodies, without the need for hybridoma-associated cell culture. These polynucleotides can then be modified using standard molecular biology techniques and expressed in cells or even in in vitro translation systems. In this manner, the cloned polynucleotides can be used to produce any amount and any type (e.g., chimeric, humanized, single chain, etc.) of conformer-specific antibody.

As discussed, herein, certain diseases are known to be associated with particular polypeptide conformers. Such diseases include, by way of example, prion diseases, neurological diseases, cancers, and viral diseases. Disease-associated conformers may be detected in blood, serum, histological samples and sections, and other biological samples, which are preferably maintained under non-denaturing conditions to preserve the folded structures of the one or more conformers present. In the case of neurological diseases, the conformers of interest may be isolated to cerebrospinal fluid or brain tissue. The antibodies produced by the methods described, herein, are useful for identifying the presence of these conformers in biological samples obtained or derived from animals.

Accordingly, using the diagnostic methods described and suggested herein, a hospital, clinic, laboratory, governmental entity, or other interested party could rapidly and efficiently screen biological samples for indicia of pathogenesis. Such screening would facilitate the rapid triage of a large number of samples, which is important in selecting treatments, isolating and handling patients, corpses, fomites, and vectors, tracking epidemics, ensuring the safety of investigative personnel, or alerting officials or their agents to public health concerns.

While such diagnostic methods are clearly of value in the fields of medicine, public health and epidemiology, they may also be useful for military and defense purposes, for example, to identify dangerous places, people, and objects. The diagnostic methods may be particularly useful for identifying pathogenic viruses in biological weapons, suspected biological weapons, or tracking their source or chain of custody.

The methods can also be used in the pharmaceutical industry and in academia to rapidly identify potential novel viruses, drug targets, research tools, or reagents.

While the clinical value of the above-identified diagnostic assays and passive immunity are readily apparent, there are also public health and military uses for the invention. Since particular conformers are now known to be associated with the virulence and/or pathogenicity of certain disease-causing organisms (e.g., Ebola virus), conformer-specific antibodies are useful for detecting the presence of pathogenic organisms in product tampering cases, in suspected biological weapons, or in terrorist attacks.

Additionally, since polypeptide conformation is known to play a role in prion-mediated diseases, conformer-specific antibodies are invaluable for identifying prion diseases in biological samples. Such diseases include but are not limited to transmissible spongiform encephalopathy (TSE) diseases, such as kuru and Creutzfeldt-Jakob disease (CJD) in humans, scrapie in sheep, transmissible mink encephalopathy (TME), and chronic wasting disease (CWD) in mule, deer, and elk, and bovine spongiform encephalopathy (BSE) in cows. The antibodies of the invention are useful for detecting these diseases in biological samples obtained from the animals suspected of having the disease, in animal feed, in slaughter houses, in processed animal products, and other biological samples that could contain a form of transmissible spongiform encephalopathy. The invention is useful for identifying such diseases both domestic animal products and in screening imported animal products. Domestically, since federal regulatory agencies have failed to ensure that animal feed is free of rendered animal products, a less burdensome approach to protecting the public health may be to screen for the presence of particular polypeptide conformers.

Furthermore these conformer-specific antibodies will allow extension of the definition of disease states, for example, if ^{Ctm}PrP is found to be a critical trigger of diabetes mellitus and of heart disease, ^{Ctm}PrP-specific antibodies will be useful in stratification, diagnosis, and possibly therapeutic intervention in these diseases as well.

The antibodies of the invention are also useful for conferring passive immunity to animals having, or at risk for contracting a disease associated with particular conformers. Passive immunity is known in the art; however, conferring passive immunity to an animal first requires the availability of appropriate antibodies, which have heretofore been difficult or impossible to produce. By providing methods for producing substantial quantities of conformer-specific antibodies, e.g., using a heterologous expression system, the present invention provides antibodies that can be used to confer passive immunity to animals, including humans, at risk for polypeptide conformer-mediated diseases.

The availability of essentially unlimited quantities of conformer-specific antibodies also permits the affinity purification of, or at least the enrichment of polypeptide samples for, particular polypeptide conformer, thereby providing a source of these conformers for vaccinating animals, producing additional conformer-specific antibodies, and for use in identifying pharmaceutical agents that bind to, inhibit, inactivate, cleave, chemically modify, or otherwise modulate the disease-causing ability of particular polypeptide conformers. As the list of diseases associated with particular polypeptide conformers continues to expand, the importance of producing quantities of the relevant conformers is readily apparent.

As discussed above, the ability to vaccinate animals is important in terms of public health, protection of humans (and other animals) against sabotage, product tampering, terrorist attacks, weapons and laboratory inspections, protection of personnel working with pathogens, protection of international health case personnel assisting epidemic-ridden regions, and otherwise affording protection against diseases caused by particular polypeptide conformers.

While this invention has been particularly shown and described with references to preferred embodiments thereof, it will be understood by those skilled in the art that various changes in form and details may be made therein without departing from the spirit and scope of the invention as defined by the appended claims.

The following Examples are provided to illustrate the application of the invention. These Examples should in no way be construed to limit the scope of the invention.

### EXAMPLES

### Example 1

### Isolation of Conformer-Specific Antibodies From a Rabbit Polyclonal Antibody Using Affinity Chromatography

Where a rabbit polyclonal antibody recognizes multiple polypeptide conformers of a subject polypeptide preparation that is enriched for a conformer of interest, antibodies that recognize a conformer of interest are identified by first depleting the polyclonal antibody of antibodies that recognize other conformers (i.e., conformers that are not of interest) then isolating antibodies that bind to the conformer of interest by forming conformer-antibody complexes.

As an initial step, the polyclonal antibody is passed over a column to which conformers that are not of interest are immobilized (i.e., bound to the column matrix) using standard chromatographic methods. The eluate (flow-through) from this chromatographic step comprises antibodies that do not bind to the conformers that are not of interest. This eluate is then passed over a column to which the conformer of interest is immobilized. Antibodies that are specific for the conformers of interest bind to the immobilized conformers of interest and are eluted (e.g., under denaturing conditions such as SDS, 2-ME, urea, guanidinium-HCl, etc.) after washing the column free of other serum components.

The eluted conformer of interest-specific antibodies are concentrated, as required, then subjected to N-terminal sequencing (e.g., via Edman degradation) to determine the amino acid residue sequence of the antigen binding portion of the H and L chains. Edman degradation and related methods are well-known in the art and are reviewed in, e.g., Fountoulakis, M.; Lahm, H. W. (1998) Hydrolysis and amino acid composition of proteins. J. Chromatogr. A. 826:109-34 and Shively, J. E. (2000) The chemistry of protein sequence analysis. EXS 88:99-117.

The identity of a sufficient number of N-terminal residue are determined to allow the design of degenerate oligonucleotide primers corresponding to polynucleotides encoding the antibody heavy and light chains of the antibodies specific for the conformer of interest. Since the oligonucleotides should be at least about 30, and preferably about 35, 40, 45, 50, or even more nucleotides in length, at least about 10, 11, 12, 13, 14, 15, 16, 17, 18, 19, 20, or more N-terminal residues are determined.

The resulting polypeptide sequences are then used to design degenerate oligonucleotide primers with which to amplify by PCR the polynucleotide coding sequences of the conformer-specific antibodies, e.g., using a genomic or cDNA library, mRNA, or to probe a library using hybridization techniques.

This procedure results in the identification of the polypeptide sequence that determines antibody specificity for the conformer of interest and yields polynucleotide reagents that can be used to identify polynucleotides encoding the relevant regions of the H and L chains of antibodies specific for the conformer of interest.

### Example 2

### Isolation of Conformer-Specific Antibodies From a Rabbit Polyclonal Antibody Using Affinity Chromatography and Native Polyacrylamide Gel Electrophoresis

Alternatively, the eluate from the first column in Example 1 is incubated in the presence of the conformer of interest. The resulting conformer-antibody complexes are resolved by native polyacrylamide gel electrophoresis. The specific conformer-antibody complexes are then excised from the gel, eluted, dissociated using denaturing conditions (e.g., SDS, 2-ME, urea, guanidinium-HCl, etc.), and resolved on a denaturing gel (e.g., SDS-PAGE), by HPLC, or by other means that allow the separation of the conformer from the antibody. Appropriate control lanes are include, e.g., those that include conformer in the absence of antibody and antibody in the absence of conformer

The bands corresponding to antibody H and L chains are then excised, eluted, and subjected to N-terminal sequencing (e.g., via Edman degradation), as above.

### Example 3

### Enrichment of Antibody-Producing Cells Prior to mRNA Isolation

The individual animal or human is immunized with a polypeptide composition comprising a conformer of interest and boosted. The conformer of interest is immobilized on a suitable support medium (e.g., Miltenyi cell isolation columns for CD antigen). Either whole blood from the immunized individual or pre-isolated B-cells (sorted, for example by marker proteins CD19, CD20, CD22, or CD72 to CD78) is passed over the column. Since B-cells, which represent about 15% of the lymphoid cell population, have the IgG inserted into the plasma membrane as part of the B-cell receptor (BCR), those clonal B-cells with conformer of interest-specific IgG bind to the columnm, allowing other blood or serum components to be washed free. The bound, viable B-cells can either be expanded prior to isolation of mRNA coding for the conformer-specific IgG or used directly as a source of mRNA encoding the heavy and light chains of the antibodies that bind the conformers of interest.

The method, or variations, thereof, increases the relative amount of mRNA that encodes the antibodies of interest, simplifying the subsequent identification and isolation of polynucleotides encoding the antibodies of interest, e.g., using library or PCR-based techniques.

### Example 4

### Vaccination of Animals Using Conformers Prepared Using Conformer-Specific Antibodies

Antibodies specific to a particular polypeptide conformer of interest, such as those prepared as described and suggested, herein, may be used to affinity purify sufficient quantities of the conformer of interest to vaccinate an animal.

Vaccination is typically performed intramuscularly (IM), using a dosage of between 1 μg and 500 μg or more of conformer and preferably between 50 μg and 300 μg of conformer. The concentration of the conformer should be between 10 μg/ml and 1 mg/ml, and preferably between 50 and 500 μg/ml. These amounts are generally suitable for a rabbit; although different quantities may also be effective. In addition, larger or smaller animals may require substantially proportionally less or more conformer.

The polypeptide conformer may be administered in a singe dose but at least one additional dose, i.e., a booster dose, is preferable. In a particular example, a booster doses is administered about 2-3 weeks following the initial dose and whenever serum antibody titers diminish.

Adjuvants may optionally be administered along with the conformer preparations. Adjuvants include but are not limited to Freund's Complete and Incomplete adjuvants, saponins, modified saponins, liposomes, mineral salts (e.g., AlK(SO₄)₂, AlNa(SO₄)₂, AlNH₄ (SO₄), silica, alum, Al(OH)₃, Ca₃ (PO₄)₂, kaolin, and carbon), polynucleotides (for example, polyIC and polyAU acids), and certain natural substances (for example, lipid A, wax D from Mycobacterium tuberculosis, as well as substances found in Corynebacterium parvum, Bordetella pertussis, and members of the genus Brucella), bovine serum albumin, diphtheria toxoid, tetanus toxoid, edestin, keyhole-limpet hemocyanin, Pseudomonal Toxin A, choleragenoid, cholera toxin, pertussis toxin, viral proteins, and eukaryotic proteins such as interferons, interleukins, or tumor necrosis factor. Such proteins may be obtained from natural or recombinant sources according to methods known to those skilled in the art. Other known immunogenic macromolecules which can be used in the practice of the invention include, but are not limited to, polysaccharides, tRNA, non-metabolizable synthetic polymers such as polyvinyl amine, polymethacrylic acid, polyvinylpyrrolidone, mixed polycondensates (with relatively high molecular weight) of 4′,4-diaminodiphenyl-methane-3,3′-dicarboxylic acid and 4-nitro-2-aminobenzoic acid or glycolipids, lipids or carbohydrates.

In the case of human, a particularly useful adjuvant is alum, particularly in the form of thixotropic, viscous, and homogeneous aluminum hydroxide gel. Other adjuvants used in humans include but are not limited to BCG (bacilli Calmette-Guerin) and Corynebacterium parvum are especially preferable

### Example 5

### Use of Conformer-Specific Antibodies to Confer Passive Immunity

To provide passive immunity to animals at risk for contracting, suspected of having, or suspected of being exposed to a conformer-mediated disease, conformer-specific antibodies prepared according to the methods of the invention are administered to the animal. In one example, up to 1 g of the antibody is administered in an intravenous manner (IV) in a suitable carrier, such as phosphate-buffered saline (PBS). Preferably, the solution is sterile filtered prior to use. Such preparations are typically administered to the animal between every other third day and multiple times per day. One or two administrations per day is preferred.

One skilled in the art will recognize that the health and body weight of the animal being conferred with passive immunity, as well as the availability of the conformer-specific antibody, will affect the amount and frequency of administration.

Methods for conferring passive immunity to an animal are described in PCT Application WO 83/00229 and also reviewed in, e.g., Casadevall, A. et al. (2004) Passive antibody therapy for infectious diseases. Nat. Rev. Microbiol. 2:695-703; Bayry, J. et al. (2004) Intravenous immunoglobulin for infectious diseases: back to the pre-antibiotic and passive prophylaxis era. Trends Pharmacol. Sci. 25:306-10; and Dunman, P. M. and Nesin, M. (2003) Passive immunization as prophylaxis: when and where will this work? Curr. Opin. Pharmacol. 3:486-96.

### Example 6

### Diagnostic Assays Using Antibodies Specific for Polypeptide Conformers

Antibodies specific for particular polypeptide conformers may be used to detect the presence of such conformers in biological samples obtained or derived from animals, including humans. Such animals may be suffering from symptoms of a disease associated with a particular conformer, be at risk of exposure to a disease mediated by a particular conformer (e.g., as a result of heredity, occupational or accidental exposure to individuals or fomites harboring infectious agents that transmit the conformer-mediated disease, presence of the animal or individual in an endemic region, consumer product tampering cases, exposure to biological weapons or components, thereof, terrorist attacks, and the like.

Biological samples include blood, lymph, saliva, sputum, cerebrospinal fluid, urine, feces, perspiration, semen, vaginal secretions, hair, skin, biopsy materials, nail scrapings, and other samples collected from animals and humans for the purpose of detecting the presence of disease-associated conformers. Preferably, such biological samples are maintained under non-denaturing condition to preserve the folded structure of the conformers of interest. However, partially denaturing conditions may also be used, for example, where the conformer of interest has a stability to one or more denaturing agents that distinguishes it from other conformers. Where the concentration of the conformer of interest a biological sample is low, it may be necessary to concentrate the sample, e.g., via dialysis, without altering the particular folded structure of the conformer that distinguishes it from other conformers of the subject polypeptide.

### Example 7

### Role of Conformers in Alcohol and Other Drug Addiction

Cannabinoid receptors are associated with alcohol and drug addiction, although polymorphisms corresponding to the addictive state/diseases have not yet been identified. At least some of these diseases are likely to involve differences in the relative amounts of certain cannabinoid conformers, which are associated with an increased tendency toward addiction.

For example, cannabinoid receptor CB1, expressed from a cloned cDNA in cell-free translation system, is produced in at least two conformers, based on the proteolytic mapping of the accessibility of certain residues (data not shown). By using the present invention to generate conformer specific antibodies it is possible to rapidly screen for the distribution of CB1 conformers in patient blood samples and thereby identify individuals at high risk for addiction.

Similarly, it is possible to separate conformers (e.g., by ion exchange chromatography), capture them in a plate assay with a non-conformer specific antibody on the plate (i.e., antibodies that recognize multiple cannabinoid receptor conformers, not only the conformer of interest), and then search for peptides or small molecules that block binding of biotinylated antibodies that are specific for the conformers of interest. Such peptides or small molecules are presumably specific for epitopes of the conformers of interest that are not present on the conformers that are not of interest. Such peptides or small molecules are then used to direct the rational design of peptidomimetic small molecule inhibitors of the conformers of interest, which can be used to treat or prevent addictions mediated by the conformers of interest.

### Example 8

### Identification of Tumor Markers in Prostatic Adenocarcinoma Cells

In this example, the above-described methods are used to identify novel tumor markers, which are not present in equivalent “normal” control samples. Such markers are useful for diagnosing tumors and for developing therapies against tumors.

In a particular example, a biological sample of prostatic adenocarcinoma cells (e.g., cells or a lysate or fraction, thereof, obtained from a human patient), along with a suitable adjuvant, is used to immunize an animal, such as a rabbit. The polyclonal serum derived from the immunized rabbit contains antibodies specific for a diverse set of immunogenic proteins, including different protein conformers, that are expressed in the tumor cells. Since most of these proteins are also expressed in normal prostate, the crude polyclonal antibodies also recognize normal prostate tissue.

Antibodies that recognize normal prostate tissues are depleted from the polyclonal sera using a column to which normal prostate epithelial cells, or a lysate or fraction, thereof, have been immobilized. Alternatively, normal prostate epithelial cells, or a lysate or fraction, thereof, are immobilized on a membrane or equivalent and incubated in the presence of the crude polyclonal antibody. Using such procedures, antibodies recognizing normal prostate are bound to the column, while antibodies specific for proteins or conformers expressed in prostatic adenocarcinoma cells remain in the column flow-through or eluant.

Antibodies specific for individual tumor-specific antigens, including conformers unique to adenocarcinoma cells, are then isolated. One approach is to separate tumor-cell proteins on a native polyacrylamide gel, transfer the polypeptides to a membrane, and incubate the membrane in the presence of the above-referenced column eluate or unbound polyclonal antibody fraction. The bound antibodies are then eluted from the membrane under denaturing conditions and the N-terminal polypeptide sequences of the component heavy and light chains is determined, as described herein.

In a related approach, prostatic adenocarcinoma cells are fixed on a histologic slide or equivalent, thereof, and the slide is incubated with the eluent, in which the antibodies present have previously been labeled with a fluorescent molecule using standard methods known in the art. The adsorbed antibodies are visualized by fluorescence microscopy then eluted and sequenced.

As a test-of-principle of the above experiment, the above procedures should yield antibodies specific for cancer-associated conformers of prostatic acid phosphatase, which have previously been identified by our laboratory group.

### Example 9

### Identification of Diagnostic Markers and/or Therapeutic Targets in Nervous System Disorders

The methods of the invention are also useful for identifying novel proteins for use as therapeutic targets and/or disease markers for central nervous system diseases such as ALS, Alzheimer's disease, Parkinson's disease, and Creutzfeldt-Jacob disease by using the specific pathologic tissue sample as the immunizing antigen.

For example, to identify protein components (including conformers) present in protein aggregates termed, “Lewy bodies,” in Parkinson's disease, sample from the diseased portion of a Parkinson's brain would be used to immunize rabbits. The resulting polyclonal serum would then be depleted or pre-adsorbed using a control sample (for example, a sample from uninvolved regions of the same brain) to remove antibodies that recognize antigens/epitopes present in normal brain tissue, including conformers present in normal brain tissue. Antibodies specific for Lewy bodies are then isolated by immobilizing Lewy body-derived samples to a column or membrane and incubating the depleted eluate in the presence of such immobilized antigens. A fluorescent label can be employed, as above. Alternatively, solubilized Lewy bodies are added to the depleted eluate samples, resulting in the formation of antibody-antigen complexes, which can be resolved by native gel electrophoresis, using appropriate controls to allow the identification of such complexes. Appropriate controls would include depleted extract incubated with normal brain tissue, Lewy bodies alone, and pre-immune serum incubated with normal brain tissue or Lewy bodies.

The antibodies specific for Lewy body antigens/epitopes is then eluted or excised as required and the heavy and light chains of such antibodies are subjected to N-terminal sequencing, as described above.

One important advantage of the present invention is greatly enhanced production efficiency. Conventional routes require screening potentially thousands of hybridomas looking for the rare conformer-specific one within 24-48 hours. The present method replaces a time-constrained procedure, with one that is not so time constrained: a practitioner of the invention can take a month or longer to obtain the results of the invention without fear of losing anything. Other advantages are that the rabbit immune repertoire is larger and more complex than that of the mouse, and rabbit antibodies are generally of higher affinity.

All references cited above are expressly incorporated by reference to the same extent as if each reference was individually incorporated.

What is claimed:

1. A method for producing an antibody specific for a polypeptide conformer, the method comprising:
isolating from a polyclonal antibody, which is produced in an animal and raised against a polypeptide preparation enriched for a polypeptide conformer of interest, an antibody specific for the polypeptide conformer of interest;
determining the polypeptide sequence of the antibody specific for the polypeptide conformer of interest;
using the polypeptide sequence to design one or more degenerate oligonucleotides comprising nucleotide sequences corresponding to polynucleotides encoding the antibody specific for the polypeptide conformer of interest; and
using the one or more degenerate oligonucleotides to identify polynucleotides encoding the antibody specific for the polypeptide conformer of interest.

2. The method of claim 1, further comprising, depleting the polyclonal antibody of antibodies that are not specific for the particular polypeptide conformer.

3. The method of claim 1, wherein isolating the polypeptide conformer of interest is performed by immunoprecipitating the antibody specific for the polypeptide conformer of interest with the conformer of interest.

4. The method of claim 1, wherein the a polypeptide composition enriched for a polypeptide conformer of interest is produced using a polynucleotide encoding a signal peptide selected from the group consisting of a heterologous signal peptide, a modified signal peptide, a chimeric signal peptide, and combinations, thereof.

5. The method of claim 1, wherein the polyclonal antibody is recovered by bleeding or exsanguination.

6. The method of claim 1, wherein the one or more degenerate oligonucleotides comprising nucleotide sequences corresponding to polynucleotides encoding the antibody specific for the polypeptide conformer of interest additionally comprise nucleotide sequences useful for inserting the oligonucleotides into a heterologous DNA sequence.

7. The method of claim 1, wherein the one or more degenerate oligonucleotides are used to identify the coding sequence of the antibody specific for the polypeptide conformer of interest from a library.

8. The method of claim 1, wherein the polynucleotide sequences encoding the antibody specific for a polypeptide conformer of interest are inserted into exogenous polynucleotides for expressing said antibody.

9. The method of claim 1, wherein the polynucleotides encoding the antibody specific for the polypeptide conformer of interest are further modified to express a modified version of said antibody selected from the group consisting of a single-chain antibody, a humanized antibody, a chimeric antibody, and an antibody fragment.

10. An antibody specific for a polypeptide conformer of interest produced by the method of claim 1.

11. The antibody of claim 10, wherein the antibody is selected from the group consisting of a single-chain antibody, a humanized antibody, a chimeric antibody, and an antibody fragment.

12. A method of using the antibody of claim 11, to identify a polypeptide conformer of interest in a sample comprising a plurality of conformers of a polypeptide.

13. The method of claim 12, wherein the sample is provided under non-denaturing conditions.

14. A method for conferring passive immunity to a patient suffering from a disease mediated by a polypeptide conformer, the method comprising administering to the patient an antibody of claim 12 along with a suitable pharmaceutical carrier.

15. A method for immunizing a patient against a disease mediated by a polypeptide conformer of interest, the method comprising administering to the patient an immunogenic amount of a polypeptide composition enriched for a polypeptide conformer of interest.

16. A kit of parts comprising an antibody of claim 12, instructions for use of the antibody in an assay to detect the presence of a polypeptide conformer of interest in a sample, and instructions for interpreting results from said assay.

17. An isolated conformer-specific antibody obtained from a polyclonal antibody raised against a polypeptide preparation comprising a conformer of interest, comprising: identifying amino acid sequences of conformer-specific antibodies present in the polyclonal antibody, using the amino acid sequences to produce polynucleotides encoding the conformer-specific antibody, expressing the conformer-specific antibody encoded by the polynucleotides in an organism or a cell free translation system, and isolating the conformer-specific antibody so expressed.

18. The isolated conformer-specific antibody obtained as in claim 17, additionally comprising depleting the polyclonal antibody of antibodies not specific for conformer of interest, then incubating the depleted polyclonal antibody in the presence of a polypeptide preparation enriched for the conformer of interest and isolating complexes comprising the conformer specific antibody and conformer of interest.

19. A conformer-specific antibody isolated from a polyclonal antibody raised against a conformer of interest by incubating the polyclonal antibody in the presence of a conformer of interest, isolating antibody-conformer of interest complexes, determining the amino acid sequence of antibodies present in the complexes, and designing polynucleotides for expression of such conformer-specific antibodies.
